(19) United States
(12) Patent Application Publication
(10) Pub. No.: US 2019/0139629 A1
(43) Pub. Date: May 9, 2019

(54) RULES ENGINE REFERENCING FOR MEDICAL DATA

(71) Applicant: GENERAL ELECTRIC COMPANY, Schenectady, NY (US)

(72) Inventors: Gordon Ian Rowlandson, Wauwatosa, WI (US); Michael Andrew Juhl, Wauwatosa, WI (US)

(21) Appl. No.: 16/230,075

(22) Filed: Dec. 21, 2018

Related U.S. Application Data
(63) Continuation-in-part of application No. 14/556,710, filed on Dec. 1, 2014.

(51) Int. Cl. G16B 45/00; G16H 10/60; G16H 70/40; G16H 50/30

(52) U.S. Cl. CPC G16B 45/00; G16H 50/30; G16H 70/40; G16H 10/60

(57) ABSTRACT

Method and system for displaying measured health data are disclosed herein. An example method includes receiving configuration for a rules engine which comprises a plurality of configurable rules. Each rule defines a link to specific information in an electronic medical record (EMR) in response to the measured health data meeting one or more criteria. The method further includes receiving the measured health data for a subject; linking the measured health data to the specific information in the EMR in response to the measured health data meeting the one or more criteria based on the configured rules engine; and displaying the measured health data and the specific information in the EMR which is linked to the measured health data.

### CROSS REFERENCE

This is a continuation-in-part (CIP) application of U.S. patent application Ser. No. 14/556,710, filed on Dec. 1, 2014, the entirety of which is incorporated herein by reference.

### BACKGROUND

The subject matter disclosed herein relates generally to techniques for displaying medical data. In diagnostic medicine, measurements may be taken for a subject, such as a patient, using various instruments. For example, an electrocardiograph (ECG) instrument may be used to record electrical activity of a heart of a patient. Many hospitals also implement electronic medical records (EMRs) employing a systematic collection of electronic health information about an individual patient or population. In many situations, some contents in the EMR may help understand the implications of the measured health data. For example, the measured health data may be impacted by the treatment received by the patient, which is generally recorded in the EMR.

### BRIEF DESCRIPTION

An embodiment relates to a method for displaying measured health data. The method comprises receiving configuration for a rules engine which comprises a plurality of configurable rules. Each rule defines a link to specific information in an electronic medical record (EMR) in response to the measured health data meeting one or more criteria. The method further comprises receiving the measured health data for a subject; linking the measured health data to the specific information in the EMR in response to the measured health data meeting the one or more criteria based on the configured rules engine; and displaying the measured health data and the specific information in the EMR which is linked to the measured health data.

Another embodiment relates to a system for displaying measured health data. The system comprises a storage device, a user interface, a processing device, and a display device. The storage device is configured to store a rules engine which comprises a plurality of configurable rules, each rule defining a link to specific information in an electronic medical record (EMR) in response to the measured health data meeting one or more criteria. The user interface is configured to receive configuration for the rules engine. The processing device is configured to receive the measured health data for a subject and link the measured health data to the specific information in the EMR in response to the measured health data meeting the one or more criteria based on the configured rules engine. The display device is configured to display the measured health data and the specific information in the EMR which is linked to the measured health data.

Still another embodiment relates to a computer-readable medium for displaying measured health data. The computer-readable medium stores processor-executable code to receive configuration for a rules engine which comprises a plurality of configurable rules, each rule defining a link to specific information in an electronic medical record (EMR) in response to the measured health data meeting one or more criteria. The medium further stores processor-executable code to receive the measured health data for a subject; link the measured health data to the specific information in the EMR in response to the measured health data meeting the one or more criteria based on the configured rules engine; and display the measured health data and the specific information in the EMR which is linked to the measured health data.

### BRIEF DESCRIPTION OF THE DRAWINGS

The present techniques will become more fully understood from the following detailed description, taken in conjunction with the accompanying drawings, wherein like reference numerals refer to like parts, in which:

FIG. 1 illustrates a block diagram illustrating a computing system configured to display health data, in accordance with an exemplary embodiment;

FIG. 2A illustrates a graphical user interface for a user to configure the rules engine, in accordance with an exemplary embodiment;

FIG. 2B illustrates a graphical user interface to render an electronic medical record (EMR) having specific data highlighted based on a rules engine reference, in accordance with an exemplary embodiment;

FIG. 3 is a flow diagram showing data flow for the rules engine, in accordance with an exemplary embodiment;

FIG. 4 is a flow chart illustrating a method for displaying health data, in accordance with an exemplary embodiment; and

FIG. 5 is a block diagram of a computer readable medium that includes modules for displaying health data, in accordance with an exemplary embodiment.

### DETAILED DESCRIPTION

In the following detailed description, reference is made to the accompanying drawings that form a part hereof, and in which is shown by way of illustration of specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the embodiments described herein.

As used herein, the terms “system,” “unit,” or “module” may include a hardware and/or software system that operates to perform one or more functions. For example, a module, unit, or system may include a computer processor, controller, or other logic-based device that performs operations based on instructions stored on a tangible and non-transitory computer readable storage medium, such as a computer memory. Alternatively, a module, unit, or system may include a hard-wired device that performs operations based on hard-wired logic of the device. Various modules or units shown in the attached figures may represent the hardware that operates based on software or hardwired instructions, the software that directs hardware to perform the operations, or a combination thereof.

Various embodiments provide techniques for animating ECG display by referencing a configurable rules engine in a medical environment. As discussed above, an electronic medical record (EMR) may employ systematic collection of electronic health information. In many situations, some contents in the EMR may help understand the implications of the measured health data, e.g., ECG data. For example, ECG may be impacted by the treatment (e.g., drugs) received by the patient, which is generally recorded in the EMR. The configurable rules engine comprises a plurality of rules each defining a link to relevant information in the EMR when the ECG data meets one or more criteria (e.g., prolonged QT, Afib, etc.). A user (e.g., a physician) can selectively enable or disable the rules based on the particular patient. The specific information in the EMR may then be linked to the ECG data based on the configured rules engine to help understand the implications of the ECG data. By displaying the ECG data along with the specific information in the EMR, the system and method disclosed herein can help a user (e.g., a physician) to assess the ECG data in context.

It should be noted that although the various embodiments are described in connection with a particular diagnostic medical instrument, such as an ECG machine, the various embodiments may be implemented in connection with other medical instruments, such as an X-ray computed tomography (x-ray CT) detector system, a Positron Emission Tomography (PET) imaging system, and the like. Additionally, the diagnostic medical instrument may be used to capture data of different subjects, including subjects other than people.

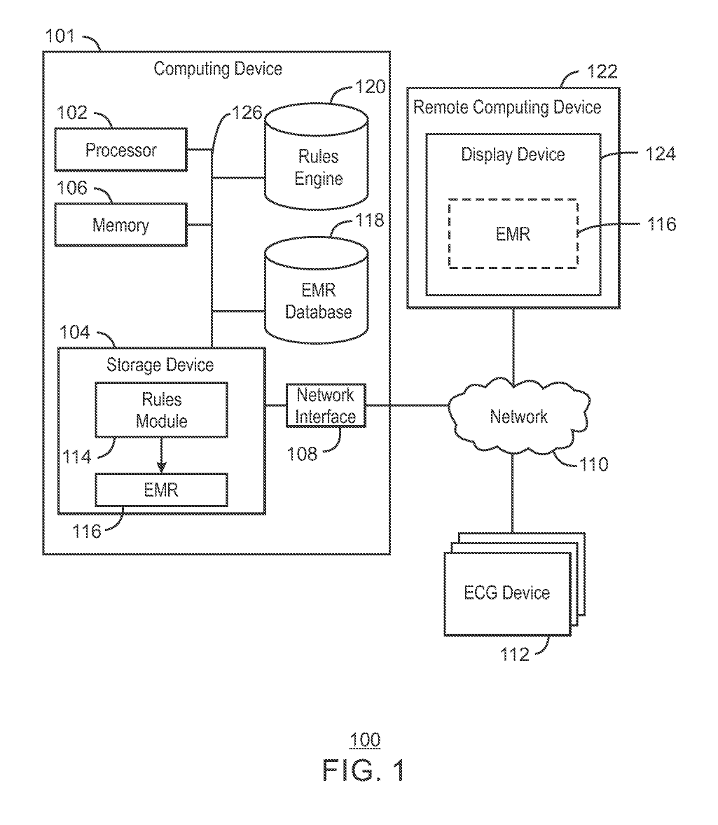

FIG. 1 illustrates a block diagram illustrating a computing system configured to reference a rules engine. The computing system 100 may include a computing device 101 having a processor 102, a storage device 104, a memory device 106, and a network interface 108. The computing device 101 may be a centralized computing device, such as server, in a medical environment, such as a hospital, a clinic, and the like. In this case, the computing device 101 may communicate, via the network interface 108, with a network 110 to receive data from one or more ECG devices 112.

The storage device 104 may be a non-transitory computer-readable medium having a rules module 114. The rules module 114 may be implemented as logic, at least partially comprising hardware logic, as firmware embedded into a larger computing system, or any combination thereof. The rules module 114 is configured to receive ECG data for a subject from the one or more ECG devices 112. The rules module 114 may be configured to identify an EMR 116 from among a plurality of EMRs stored in an EMR database 116. A rules engine 120 may be referenced by the rules module 114. The rules engine 120 may be disposed within the computing device 101 or communicatively coupled to the computing device 101 via the network 110. Similarly, the EMR database 118 may be stored locally on the computing device 101 or be communicatively available to the computing device 101 via the network 110. In any case, the rules engine 120 comprises multiple configurable rules each defining when ECG data is to be linked to specific information in the identified EMR 116.

In some embodiments, the rules module 112 may be configured to modify the EMR 116 to be presented at a remote computing device 122 having a display device 124 via the network 110, as indicated by the dashed box 116. Modifications to the EMR 116 are based on various rules defined in the rules engine. For example, a QT value above a certain threshold may be defined as abnormal by a rule in the rules engine 120. If this rule is enabled, the rules module 114 may highlight QT data in the EMR 116 rendered at the display device 124. In other words, the rules module 114 may be configured to link ECG data to specific information in the EMR 116 if one or more criteria set in enabled rule(s) are met.

For example, the rules engine 120 may define that when one or more criteria are met, additional information from the EMR 116 would be displayed along with the ECG data, including, for example, how long ago the ECG data was measured, electrolyte levels measured, drugs taken by the patient, and so on. By providing specific information in the EMR along with ECG data based on the rules engine, relevant data may be presented to a viewer to help the viewer understand the ECG data. For example, if the abnormal QT/QTc value was acquired past a certain time period threshold, then the abnormal QT/QTc value may not be relevant.

In some embodiments, rules engine 120 maintains a table for the various rules. Examples are illustrated by Tables 1A and 1B below:

TABLE 1A

| | | Generic Logic | Enable | Access | |
|---|---|---|---|---|---|
| | Rule Title | Applied | Rule | Rights | ECG Inputs |
| 1 | Long QT/QTc - | When user points to | Yes/No | All Users | Current ECG |
| | Simple Help | highlighted value, an | | | |
| | | advisory message | | | |
| | | appears | | | |
| 2 | Long QT/QTc - | When user points to | Yes/No | All Users | Current ECG |
| | Simple Help with | highlighted value, an | | | |
| | Superimposed | advisory message | | | |
| | Waveforms | appears | | | |
| 3 | Long QT/QTc - | When user points to | Yes/No | Physician | Current ECG |
| | Portal to | highlighted value, | | class level I | |
| | Electrolytes | help message appears | | | |
| | | along with | | | |
| | | supplemental | | | |
| | | information | | | |
| 4 | Long QT/QTc - | When user points to | Yes/No | Physician | Current ECG |
| | Portal to | highlighted value, | | class level | |
| | Electrolytes and | help message appears | | II | |
| | Drug interactions | along with | | | |
| | | supplemental | | | |
| | | information | | | |
| 5 | Afib | When user points to | Yes/No | All Users | Current ECG, |
| | | highlighted value, an | | | Confirmed |
| | | advisory message | | | |
| | | appears | | | |
| 6 | Afib -CHADs | When user points to | Yes/No | Physician | Current ECG, |
| | Score | highlighted value, | | level III | Confirmed |
| | | help message appears | | | |
| | | along with | | | |
| | | supplemental | | | |
| | | information | | | |

In Table 1A, six example rules are illustrated. Table 1B below include more details associated with each rule.

TABLE 1B

| | Required | | | | | | |
|---|---|---|---|---|---|---|---|
| | HL7 Inputs | | | | | | |
| | From EMR | Highlighted | | | | | |
| | with | Critical | | | | | |
| | Criterion for | Value and | | Help | Supplemental | Supplemental | Supplemental |
| | Timeliness | Rule | Action | Message | Waveform | Table | Table |
| 1 | None | QT/QTc > 460 ms | Display help | A QT/QTc of | | | |
| | | | message | this length is | | | |
| | | | | considered of | | | |
| | | | | high risk. | | | |
| | | | | See | | | |
| | | | | following | | | |
| | | | | Web Site for | | | |
| | | | | specific | | | |
| | | | | drugs and | | | |
| | | | | guidance on | | | |
| | | | | actions | | | |
| 2 | None | QT/QTc > 460 ms | Display help | A QT/QTc of | Superimposed | | |
| | | | message and | this length is | beats | | |
| | | | supplemental | considered of | with tic | | |
| | | | waveform | high risk. | marks | | |
| | | | | See | | | |
| | | | | following | | | |
| | | | | Web Site for | | | |
| | | | | specific | | | |
| | | | | drugs and | | | |
| | | | | guidance on | | | |
| | | | | actions | | | |
| 3 | Potassium | QT/QTc > 460 ms | Display help | A QT/QTc of | Superimposed | Electrolytes | |
| | (24 hours), | | message, | this length is | beats | | |
| | Calcium (12 | | supplemental | considered of | with tic | | |
| | hours), | | waveform | high risk. | marks | | |
| | Sodium (6 | | and table | See | | | |
| | hours) | | | following | | | |
| | | | | Web Site for | | | |
| | | | | specific | | | |
| | | | | drugs and | | | |
| | | | | guidance on | | | |
| | | | | actions | | | |
| 4 | Potassium | QT/QTc > 460 ms | Display help | A QT/QTc of | Superimposed | Electrolytes | Drugs |
| | (24 hours), | | message, | this length is | beats | | known |
| | Calcium (12 | | supplemental | considered of | with tic | | to |
| | hours), | | waveform | high risk. | marks | | prolong |
| | Sodium (6 | | and table | See patient | | | QT |
| | hours), | | | information | | | |
| | Drugs | | | related to this | | | |
| | | | | risk. | | | |
| 5 | | Afib is | Display help | Afib is | | | |
| | | Present | message | correlated | | | |
| | | | | with stroke. | | | |
| | | | | Considerable | | | |
| | | | | follow up | | | |
| 6 | Values | Afib is | Display help | Patient does | | CHADS | |
| | related to | Present and | message and | not appear to | | score | |
| | CHADS | Not on | supplemental | be on anti- | | | |
| | | Anti- | table | coagulants. | | | |
| | | Coagulants | | CHADS | | | |
| | | | | score > 2 is | | | |
| | | | | considered | | | |
| | | | | high risk | | | |

As illustrated in Table 1A and continuing on Table 1B, the various rules may be enabled/disabled by users with appropriate access, from the computing device 101 or from the remote computing device 122 via network 110. For example, all users can enable or disable the rules of “Long QT/QTc—Simple Help,” “Long QT/QTc—Simple Help with Superimposed Waveforms,” and “Afib.” It requires at least Physician class level I to enable or disable the rule of “Long QT/QTc—Portal to Electrolytes.” It requires at least Physician class level II to enable or disable the rule of “Portal or Electrolytes and Drug interactions.” And it requires at least Physician class level III to enable to disable the rule of “Long QT/QTc—Portal to Electrolytes and Drug Interactions” and “Afib—CHADs Score.”

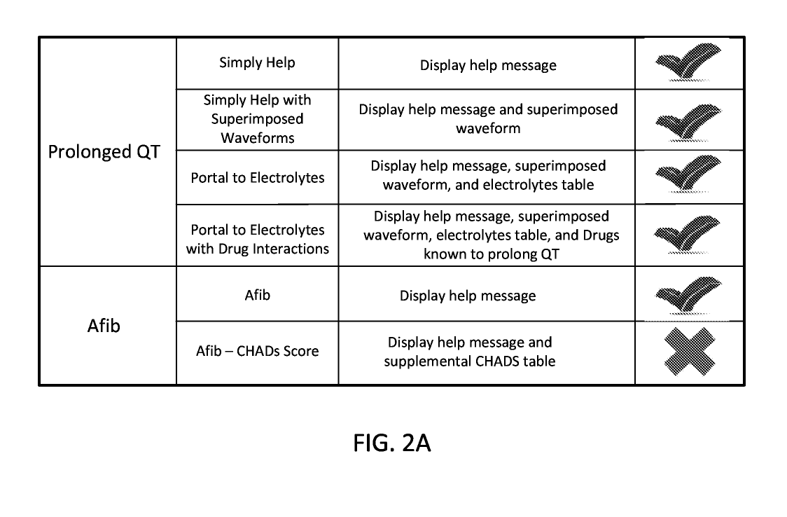

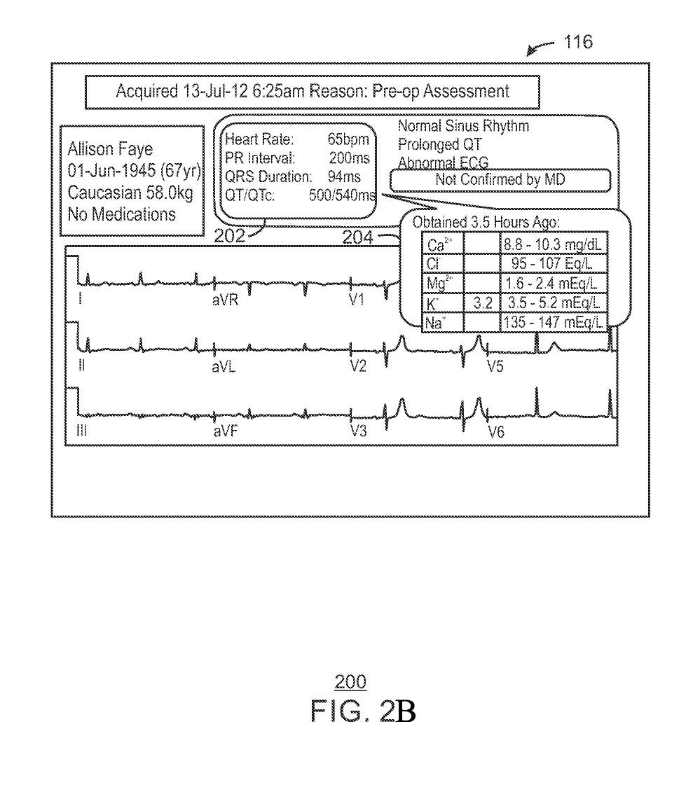

FIG. 2A illustrates a graphical user interface for configuring the rules, in accordance with an exemplary embodiment. The graphical user interface may be rendered at the computing device 101 and/or the remote computing device 122. A user (e.g., a physician) may enable (✓) or disable ( [symbol] ) a rule from the graphical user interface based on the situation of the patient.

As an example, clinical guidelines state a QT (corrected for heart rate) that exceeds 500 ms or increases by 60 ms should identify a patient at risk. QT may be automatically measured (and corrected for heart rate) by, for example, 12-lead ECG monitor. QT can be prolonged for a number of reasons. Without the rules engine, a physician has to manually gather this information from the EMR to assess the actual level of risk of a long QT for a particular patient and the next steps to reduce the risk. With the technology disclosed herein, a physician can decide which information is relevant and the relevant information can be retrieved from the EMR automatically. For example, the physician may configure the rules engine based on how recent the ECG was acquired. If the ECG is over 6 hours old, it may not be relevant. The physician may disable the rules related to prolonged QT in this situation. Another factor may be what stage the patient is at. If it is post-cardiac surgery, QT would be distorted for a number of reasons and should not be a concern. In this situation, the physician may disable the rules related to prolonged QT.

As another example, atrial fibrillation (Afib) can be detected. In some situations, once the Afib is detected, anti-coagulants are administered to the patient. In other situations, anti-coagulants are not administered. There are complicated formulas that physicians use to determine the risk/benefit of using an anti-coagulant, one of which is the CHADS(s) score. Therefore, a physician might want to enable the rule of “Afib—CHADs score” when anti-coagulant is not used, while disable the rule when anti-coagulant is used.

It should be understood that the six rules explained above are for illustration not for limitation. The rules engine 120 may store any appropriate rules for a user to configure. For example, there may be rules related to and configured for the reason why ECG was ordered and acquired. If the ECG is for a pre-operation evaluation, a rule may say “displaying a separate interpretation identifying the risk of the patient undergoing surgery.” When a physician enables the rule, the ECG data and information in the EMR may be analyzed, the risk of surgery may be assessed and displayed to the physician based on the analysis. If the ECG is acquired because of palpitations, a rule may say “emphasizing the rhythm in ECG presentation, including where the computer detected artificial pacing, P-waves,” etc. If the ECG is acquired post cardiac surgery, a rule may say “analyzing ECG for infarctions,” which may be configured by hours/days, and so on.

FIG. 2B illustrates a graphical user interface to render an EMR having specific data highlighted based on the configured rules engine. As discussed above, a rules engine, such as the rules engine 120, may define a QT period, as well as a corrected QT (QTc) period above a predetermined threshold (e.g., 500 ms) to be associated with a health risk. Therefore, as indicated in FIG. 2B, QT/QTc data above the predetermined threshold may be highlighted in a graphical user interface 200, as indicated at 202. The graphical user interface 200 may be configured to render an EMR, such as the EMR 116, in a display device, such as a display device 124 of the remote computing device 122 of FIG. 1.

A plurality of rules may be implemented by the rules engine to display information that is relevant for a viewer to assess the ECG data. For example, if a viewer clicks on the abnormal QT/QTc value 202, additional operations may be performed beyond the display of additional information 204, based on the configured rules engine. These additional operations may include, for example, a more detailed view of an ECG waveform than a wave form 206 illustrated in FIG. 2 (if the rule of “Simply Help with Superimposed Waveforms” is enabled), a list of electrolyte values highlighting those that are outside normal limits (if the rule of “Portal to Electrolytes” is enabled), lists of drugs known to prolong a QT period that the patient is currently taking as indicated in the EMR (if the rules of “Portal to Electrolytes with Drug Interactions” is enabled), and the like. Rules may be applied for other types of ECG data such as atrial fibrillation (AFIB), an aggregate metric, and the like, as indicated in Tables 1A and 1B. An example of an aggregate metric may include a CHADS score wherein congestive heart failure, hypertension, age, diabetes mellitus, prior stroke or thromboembolism, are used in aggregate to estimate risk of a medical condition such as a stroke in a patient (if the rule of “Afib—CHADs Score” is enabled).

It is important to note that although the system 100 illustrates computing devices 101 and 122, as being distributed, other configurations are possible and considered by the techniques described herein. For example, the computing device 101 may also include a display device, either integrated or peripheral to the computing device 101, to display the EMR 116 having modified information. In any case, the particular implementation will reference a rules engine to determine when specific ECG data is to be linked with a given EMR. Further examples of when specific ECG data is linked with a given EMR are described in more detail below.

Referring back to FIG. 1, the processor 102 may be a main processor that is adapted to execute the stored instructions. The processor 102 may be a single core processor, a multi-core processor, a computing cluster, or any number of other configurations. The processor 102 may be implemented as Complex Instruction Set Computer (CISC) or Reduced Instruction Set Computer (RISC) processors, x86 Instruction set compatible processors, multi-core, or any other microprocessor or central processing unit (CPU).

The memory device 106 can include random access memory (RAM) (e.g., static RAM, dynamic RAM, zero capacitor RAM, Silicon-Oxide-Nitride-Oxide-Silicon, embedded dynamic RAM, extended data out RAM, double data rate RAM, resistive RAM, parameter RAM, etc.), read only memory (ROM) (e.g., Mask ROM, parameter ROM, erasable programmable ROM, electrically erasable programmable ROM, etc.), flash memory, or any other suitable memory systems. The main processor 102 may be connected through a system bus 126 (e.g., PCI, ISA, PCI-Express, etc.) to the network interface 108. The network interface 108 may enable the computing device 101 to communicate, via the network 112, with the ECG devices 112 and the remote computing device 122.

In embodiments, the remote computing device 122 may render images at the display device 124. The display device 108 may an integrated component of the remote computing device 122, a remote component such as an external monitor, or any other configuration enabling the remote computing device 122 to render a graphical user interface. As discussed above, and in more detail below, a graphical user interface rendered at the display device 124 may be used to render the EMR 116 based on a reference to the rules engine 120. The graphical user interface may also be used for a user (e.g., a clinician) to configure the rules engine 120.

The block diagram of FIG. 1 is not intended to indicate that the computing device 101 is to include all of the components shown in FIG. 1. Further, the computing device 101 may include any number of additional components not shown in FIG. 1, depending on the details of the specific implementation.

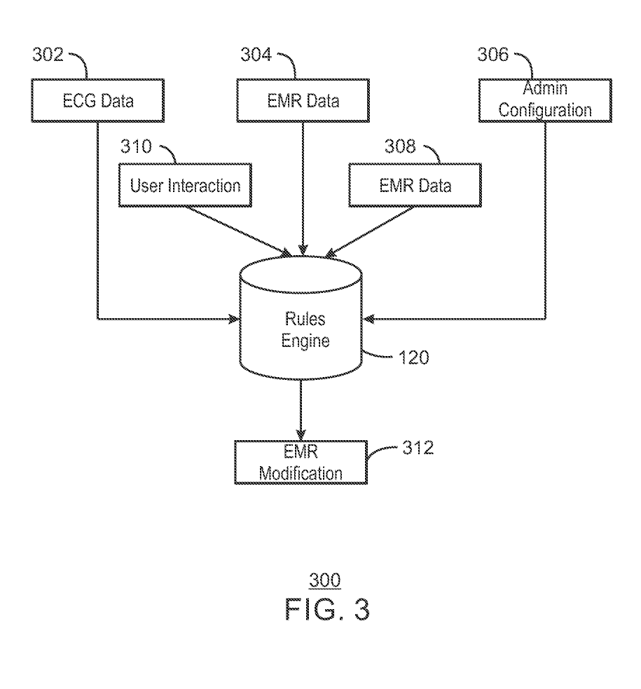

FIG. 3 is a flow diagram showing data flow for the rules engine, in accordance with an exemplary embodiment. As illustrated in FIG. 3, inputs to a rules engine, such as the rules engine 120 of FIG. 1, may include ECG data 302, EMR data 304, an administrator's configuration 306, and EMR data 308. Further, in some cases, a user's interaction with the data may be used to dynamically change what is presented in the EMR. Based on these various inputs, the rules engine 120 outputs an EMR modification 312.

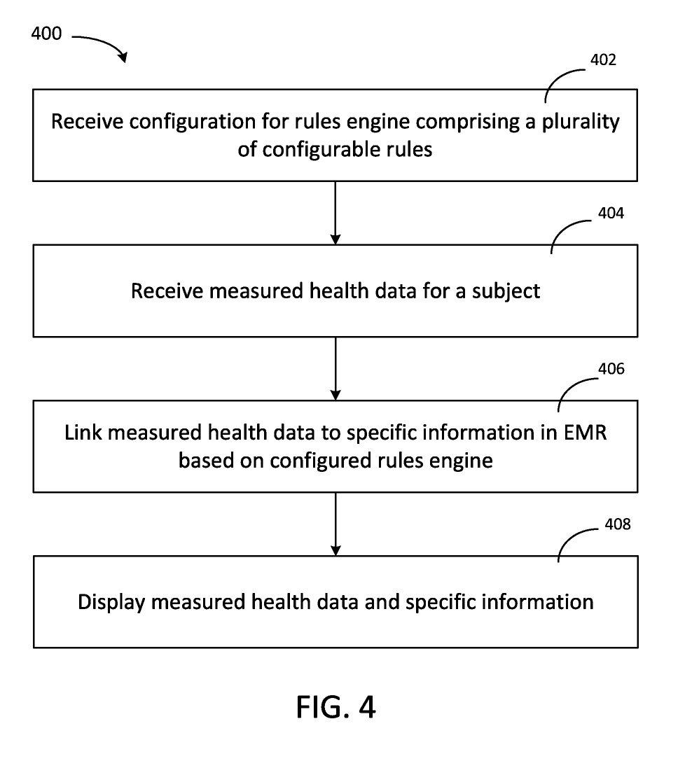

FIG. 4 is a flow chart illustrating a method for displaying health data. The method begins at operation 402 wherein configuration for rules engine is received. The rules engine (e.g., rules engine 120) comprises a plurality of rules (e.g., the 6 rules discussed above), each rule defining a link to specific information in EMR in response to one or more criteria (e.g., prolonged QT, Afib, etc.) are met. The rules may be configured (i.e., enabled/disabled) by a user through a user interface (as shown in FIG. 2A, for example). A user may configure the rules based on context related to the capture of the ECG data for the subject, based on a medical condition of the subject, or any combination thereof. For example, the context may include whether or the not subject is being measured before or after a medical operation, whether the subject is on any medication affecting an ECG measurement result, what area of a hospital the patient is in, how recent the ECG data is, and so on.

At operation 404, measured health data (e.g., ECG data) is received for a subject. At operation 406, measured health data is linked to specific information (e.g., superimposed waveform, electrolytes information of the subject, drugs taken by the subject, etc.) in EMR in response to the one or more criteria being met based on the configured rules engine. At operation 408, the specific information in EMR is displayed along with the ECG data (as shown in FIG. 2B, for example).

The method 400 may include additional operations not illustrated in FIG. 4. For example, the method 400 may include creating a table (e.g., Tables 1A and 1B) to maintain the plurality of rules in the rules engine. Each rule may define a link between ECG data and specific information in the EMR. The table may also record the enabled/disabled status of each rule.

The method 400 may further include analyzing the measured data and the specific information in the EMR and rendering the analysis along with the measured data and the specific information. For example, If the ECG is for a pre-operation evaluation, a rule may say “displaying a separate interpretation identifying the risk of the patient undergoing surgery.” When a physician enables the rule, the ECG data and information in the EMR may be analyzed, the risk of surgery may be assessed and displayed to the physician based on the analysis.

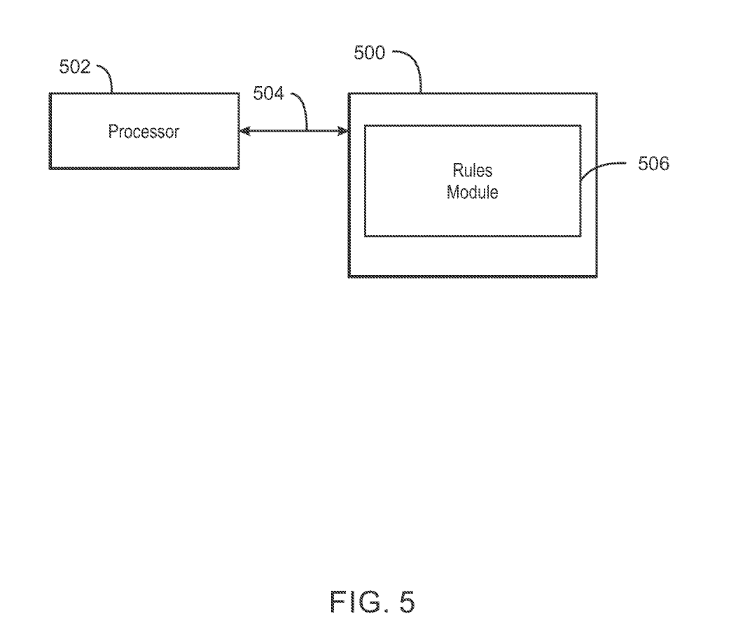

FIG. 5 is a block diagram of a computer readable medium that includes modules for rules engine referencing. The computer readable medium 500 may be a non-transitory computer readable medium, a storage device configured to store executable instructions, or any combination thereof. In any case, the computer-readable medium is not configured as a carry wave or a signal.

The computer-readable medium 500 includes code adapted to direct a processor 502 to perform actions. The processor 502 accesses the modules over a system bus 504. A rules module 506 may be configured to receive electrocardiograph (ECG) data for a subject and identify an electronic medical record (EMR) associated with the subject. The rules module 506 may also be configured to reference a rules engine comprising a plurality of configurable conditions defining when specific ECG data is to be linked to the EMR, and link the specific ECG data to the EMR if one or more of the plurality of configurable conditions are met.

While the detailed drawings and specific examples given describe particular embodiments, they serve the purpose of illustration only. The systems and methods shown and described are not limited to the precise details and conditions provided herein. Rather, any number of substitutions, modifications, changes, and/or omissions may be made in the design, operating conditions, and arrangements of the embodiments described herein without departing from the spirit of the present techniques as expressed in the appended claims.

This written description uses examples to disclose the techniques described herein, including the best mode, and also to enable any person skilled in the art to practice the techniques described herein, including making and using any devices or systems and performing any incorporated methods. The patentable scope of the techniques described herein is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A method for displaying measured health data, comprising:
receiving configuration for a rules engine which comprises a plurality of configurable rules, each rule defining a link to specific information in an electronic medical record (EMR) in response to the measured health data meeting one or more criteria;
receiving the measured health data for a subject;
linking the measured health data to the specific information in the EMR in response to the measured health data meeting the one or more criteria based on the configured rules engine; and
displaying the measured health data and the specific information in the EMR which is linked to the measured health data.

2. The method of claim 1, wherein receiving configuration for the rules engine includes receiving input from a user, which selectively enables or disables each of the plurality of configurable rules.

3. The method of claim 1, wherein the measured health data is electrocardiograph (ECG) data.

4. The method of claim 3, wherein the specific information in the EMR includes a superimposed waveform of the subject.

5. The method of claim 3, wherein the specific information in the EMR includes electrolytes information of the subject.

6. The method of claim 3, wherein the specific information in the EMR includes information of drugs taken by the subject.

7. The method of claim 3, wherein the one or more criteria includes that the ECG data shows prolonged QT.

8. The method of claim 3, wherein the one or more criteria includes that the ECG data shows Afib.

9. The method of claim 1, further comprising creating a table to maintain the plurality of configurable rules in the rules engine.

10. The method of claim 1, further comprising:
analyzing the measured data and the specific information in the EMR; and
rendering the analysis along with the measured data and the specific information.

11. A system for displaying measured health data, comprising:
a storage device configured to store a rules engine which comprises a plurality of configurable rules, each rule defining a link to specific information in an electronic medical record (EMR) in response to the measured health data meeting one or more criteria;
a user interface configured to receive configuration for the rules engine;
a processing device configured to:
receive the measured health data for a subject; and
link the measured health data to the specific information in the EMR in response to the measured health data meeting the one or more criteria based on the configured rules engine; and
a display device configured to display the measured health data and the specific information in the EMR which is linked to the measured health data.

12. The system of claim 11, wherein the measured health data is electrocardiograph (ECG) data.

13. The system of claim 12, wherein the specific information in the EMR includes at least one of superimposed waveform of the subject, electrolytes information of the subject, and information of drugs taken by the subject.

14. The system of claim 12, wherein the one or more criteria includes that the ECG data shows prolonged QT.

15. The system of claim 12, wherein the one or more criteria includes that the ECG data shows Afib.

16. The system of claim 11, wherein the processing device is further configured to analyze the measured data and the specific information in the EMR, and the display device is further configured to display the analysis along with the measured data and the specific information.

17. A non-transitory computer-readable medium for rules engine referencing, the computer-readable medium storing processor-executable code to:
receive configuration for a rules engine which comprises a plurality of configurable rules, each rule defining a link to specific information in an electronic medical record (EMR) in response to the measured health data meeting one or more criteria;
receive the measured health data for a subject;
link the measured health data to the specific information in the EMR in response to the measured health data meeting the one or more criteria based on the configured rules engine; and
display the measured health data and the specific information in the EMR which is linked to the measured health data.

18. The medium of claim 17, wherein the measured health data is electrocardiograph (ECG) data, and the specific information in the EMR includes at least one of superimposed waveform of the subject, electrolytes information of the subject, and information of drugs taken by the subject.

19. The medium of claim 18, wherein the one or more criteria includes at least one of that the ECG data shows prolonged QT and that the ECG data shows Afib.

20. The medium of claim 17, further comprising processor-executable code to:
analyze the measured data and the specific information in the EMR; and
render the analysis along with the measured data and the specific information.
